(19) United States
(12) Patent Application Publication
(10) Pub. No.: US 2006/0263442 A1
(43) Pub. Date: Nov. 23, 2006

(54) Anticancer drug using deep sea water

(75) Inventors: Yoshinobu Kozuka, Kanazawa-shi (JP); Akira Tuji, Kanazawa-shi (JP)

Correspondence Address: OLIFF & BERRIDGE, PLC, P.O. BOX 19928, ALEXANDRIA, VA, 22320, US

(73) Assignee: Yoshinobu KOZUKA, Kanazawa-shi (JP)

(21) Appl. No.: 11/410,957

(22) Filed: Apr. 26, 2006

Related U.S. Application Data
(63) Continuation of application No. PCT/JP03/13737, filed on Oct. 27, 2003.

(51) Int. Cl. A61K 33/14; A61K 33/00

(52) U.S. Cl. 424/600; 424/680

(57) ABSTRACT

The deep sea water collected from a depth of about 300 meters in the Sea of Japan was injected into a concentrator provided with a rotary drum type decompression chamber, and concentrated under reduced pressure by steam heating from the periphery. The resulting concentrate was used as an anticancer drug stock solution, and mice were treated using a test solution obtained by diluting the stock solution 200 times. As a result, the test solution exhibited anticancer effects on transplanted HeLa cells.

### CROSS REFERENCE TO RELATED APPLICATION

This application is a continuation of International Patent Application No. PCT/JP2003/013737, having an international filing date of Oct. 27, 2003, which designated the United States, the entirety of which is incorporated herein by reference.

### BACKGROUND OF THE INVENTION

The present invention relates to an anticancer drug using sea water collected from the deep sea (hereinafter called “deep sea water”) as the raw material.

Sea water in the deep sea at a depth of 200 to 300 meters or more contains minerals and sea bacteria differing from those of normal sea water. Therefore, the deep sea water has been increasingly used not only for drinking water, but also for various types of food processing.

In this case, it is necessary to concentrate or desalinate the deep sea water.

As a method for desalinating or concentrating the deep sea water, an ion-exchange method (JP-A-2001-211864) and an electrolysis method (JP-A-2000-23646) have been proposed. However, these methods do not allow the deep sea water to fully exhibit the original efficacy.

### BRIEF SUMMARY OF THE INVENTION

According to one aspect of the invention, there is provided an anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %.

According to another aspect of the invention, there is provided an anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %,

wherein the concentrated sea water is obtained by a method of concentrating the deep sea water comprising steps of:

injecting the deep sea water into a decompression chamber;

evaporating water in the deep sea water by heating the deep sea water in the decompression chamber by supplying steam at an atmospheric pressure superheated to a temperature equal to or higher than a boiling temperature by high-frequency heating to a steam chamber formed around the decompression chamber with a heat conducting wall interposed therebetween; and

obtaining the concentrated sea water remaining in the decompression chamber.

According to another aspect of the invention, there is provided an anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %,

wherein the concentrated sea water is obtained by a method of concentrating the deep sea water comprising steps of:

injecting the deep sea water into a decompression chamber;

obtaining steam by spraying air, which passes through a high-frequency coil, with water.

evaporating water in the deep sea water by heating the deep sea water in the decompression chamber by supplying the steam at an atmospheric pressure superheated to a temperature equal to or higher than a boiling temperature to a steam chamber formed around the decompression chamber with a heat conducting wall interposed therebetween; and

obtaining the concentrated sea water remaining in the decompression chamber.

According to a further aspect of the invention, there is provided an anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %,

wherein the concentrated sea water is obtained by a method of concentrating the deep sea water comprising steps of:

injecting the deep sea water into a decompression chamber;

obtaining steam by passing saturated steam, which is produced by a boiler, through a steam pipe wound with a high-frequency coil.

evaporating water in the deep sea water by heating the deep sea water in the decompression chamber by supplying the steam at an atmospheric pressure superheated to a temperature equal to or higher than a boiling temperature to a steam chamber formed around the decompression chamber with a heat conducting wall interposed therebetween; and

obtaining the concentrated sea water remaining in the decompression chamber.

### BRIEF DESCRIPTION OF THE SEVERAL VIEWS OF THE DRAWING

FIG. 1 shows component analysis results of a deep sea water concentrate (anticancer drug stock solution) and a 200-fold diluted solution (anticancer drug test solution) subjected to an anticancer effect investigation;

FIG. 2 is a graph 1 showing the age (weeks) and changes in weight of each mouse in each group, in which data provided by Japan SLC, Inc. is plotted by “x”;

FIG. 3 contains graphs 2, 3, and 4 showing tumor volume measurement data for each group (n=5 to 6);

FIG. 4 is a graph 5 showing changes in the average value and the standard deviation (n=5 to 6) of the tumor volume of the first group (anticancer drug test solution) and the third group (normal water);

FIG. 5 is a schematic sectional side view showing an embodiment of a desalinating and condensing apparatus for obtaining an anticancer drug stock solution according to the invention;

FIG. 6 is a vertical sectional view of a rotating drum of the apparatus of FIG. 5; and

FIG. 7 is a schematic sectional side view showing another example of a superheated steam generating apparatus.

### DETAILED DESCRIPTION OF THE EMBODIMENT

The inventors of the invention have conducted various studies on pharmaceutical effects of deep sea water. As a result, the inventors have found that the deep sea water exhibits anticancer effects.

An investigation method and investigation results for the anticancer effects of the deep sea water on mice are described below.

Preparation of Deep Sea Water

The deep sea water was collected from a depth of about 300 meters in the Sea of Japan offshore from Uchiura-machi, Ishikawa, Japan.

The collected deep sea water was injected into a concentrator provided with a rotary drum type decompression chamber, and concentrated under reduced pressure by steam heating from the periphery.

The decompression conditions affect the steam heating temperature, concentration, and concentration time. The deep sea water easily boils at a low temperature by increasing the degree of vacuum in the decompression chamber. However, if the deep sea water boils too violently, the components of the deep sea water may be mixed into the distilled water.

The degree of vacuum may be relatively decreased in order to increase the concentration rate.

Steam was used as the heat source because the heating temperature can be prevented from being increased to a large extent and the decompression chamber can be efficiently and uniformly heated from the outside over the peripheral portion.

When high-frequency heating saturated steam obtained from a boiler or the like by causing the saturated steam to pass through a pipe provided with a wound high-frequency coil, heated steam at 200° C. or more is obtained at normal pressure. It is efficient to use such heated steam as the heat source of the decompression chamber.

Note that the applicants of the invention have filed patent applications in Japan (Japanese Patent Application No. 2002-112339) and in the U.S.A (U.S. patent application Ser. No. 10/254,503) which are directed to the above concentration method and apparatus. The concentration method and apparatus are described later.

The above-described example in which heated steam is used as the heat source when concentrating the deep sea water by heating under reduced pressure is an example of a process which allows the deep sea water to be efficiently heated.

Note that the heat source is not limited thereto. For example, electricity, gas, or the like may also be used.

The deep sea water concentrated to have a sodium chloride content of about 15 to 18 mass % was used as an anticancer drug stock solution.

In this specification, deep sea water with a sodium chloride content of 15 mass % or more is called high-concentration deep sea water.

A solution obtained by diluting the stock solution about 200 times was used for the investigation of anticancer effects on nude mice.

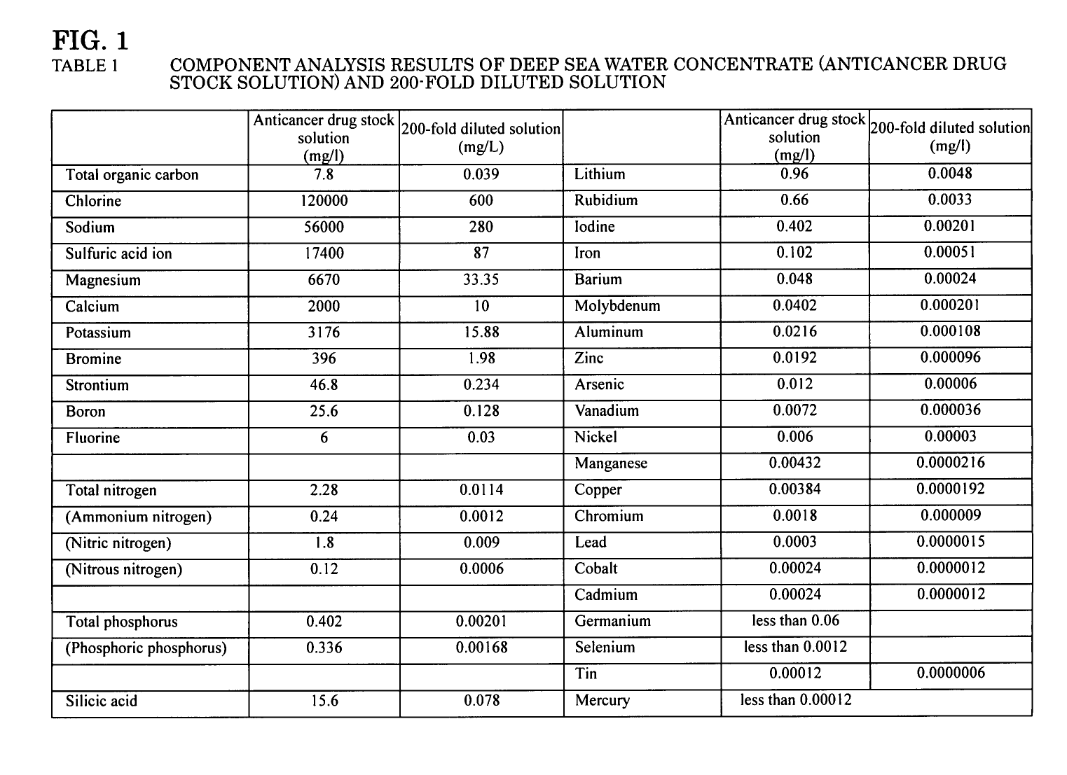

FIG. 1 (Table 1) shows the compositions of the anticancer drug stock solution and the 200-fold diluted solution.

The pH of the anticancer drug stock solution was 8.3.

As shown in FIG. 1, the stock solution contains a larger amount of various minerals in comparison with normal sea water.

Investigation of Anticancer Effects

In this specification, a solution obtained by concentrating the deep sea water is called an anticancer drug stock solution, and a solution obtained by diluting the anticancer drug stock solution about 200 times with deionised water is called an anticancer drug test solution.

Three groups of nude mice (Japan SLC, Inc., BALB/cSlc-nu/nu) were individually treated and investigated.

The mice in the first group were administered the anticancer drug test solution.

The mice in the second group were administered the anticancer drug test solution sterilized by filtration.

The mice in the third group were administered normal breeding water (hereinafter called “normal water”).

The anticancer drug test solution was obtained by diluting the anticancer drug stock solution 200 times with deionised water which was sterilized by filtration (filter: “Mediakap-2 ME2M-020-18S” manufactured by SPECTRUM, pump: “ROLLER PUMP RP-NE3” manufactured by FURUE SCIENCE Co., Ltd.), and stored at 4° C. 200 ml of the anticancer drug test solution was aseptically placed in each empty water bottle on a clean bench in an anteroom to a breeding room. Two water bottles were placed in each cage of the first group.

The anticancer drug test solution sterilized by filtration was obtained by sterilizing the anticancer drug stock solution diluted 200 times with deionised water by filtration.

The resulting anticancer drug test solution was stored at 4° C., and placed in a water bottle on a clean bench in the same manner as for the first group. Two water bottles containing 200 ml of the anticancer drug test solution were placed in each cage of the second group.

Two water bottles containing 200 ml of normal water were placed in each cage of the third group.

The cage was cleaned once a week. The water intake was measured and recorded when cleaning the cage.

The mice in each group were administered food “CE-2” (manufactured by CLEA Japan, Inc.) and kept for 10 days.

After keeping the mice for 10 days, HeLa cells were transplanted into the mice. The weight and the tumor size of each mouse were measured twice a week. The tumor tissues and the blood were collected 35 days after the tumor cell transplantation.

(HeLa Cell Hypodermic Transplantation Method)

HeLa cells (Health Science Research Resources Bank (HSRRB)) were removed in an incubation room using a rubber policeman, placed in a centrifugation tube, and centrifuged at 200 g at 4° C. for five min.

After removing the supernatant liquid, a medium was prepared using a DMEM (+) (“D5796” manufactured by SIGMA) so that the amount of cells was 2×107 cells/ml.

The medium was cooled with ice and moved to the animal room. The following operations were then performed in the order of the first group, the second group, and the third group.

Absorbent cotton was placed in a lidded anesthesia glass container and soaked with diethyl ether (“15402-35” manufactured by Nacalai Tesuque) to anesthetize the mice.

The cell suspension was drawn into a syringe (“SS-02Sz” manufactured by TERUMO Co., Ltd.). After installing a 25G needle (“NN-2525R” manufactured by TERUMO Co., Ltd.), 0.1 ml (2×106 cells) of the cell suspension was transplanted hypodermically to the right abdomen of the mouse.

Investigation Results

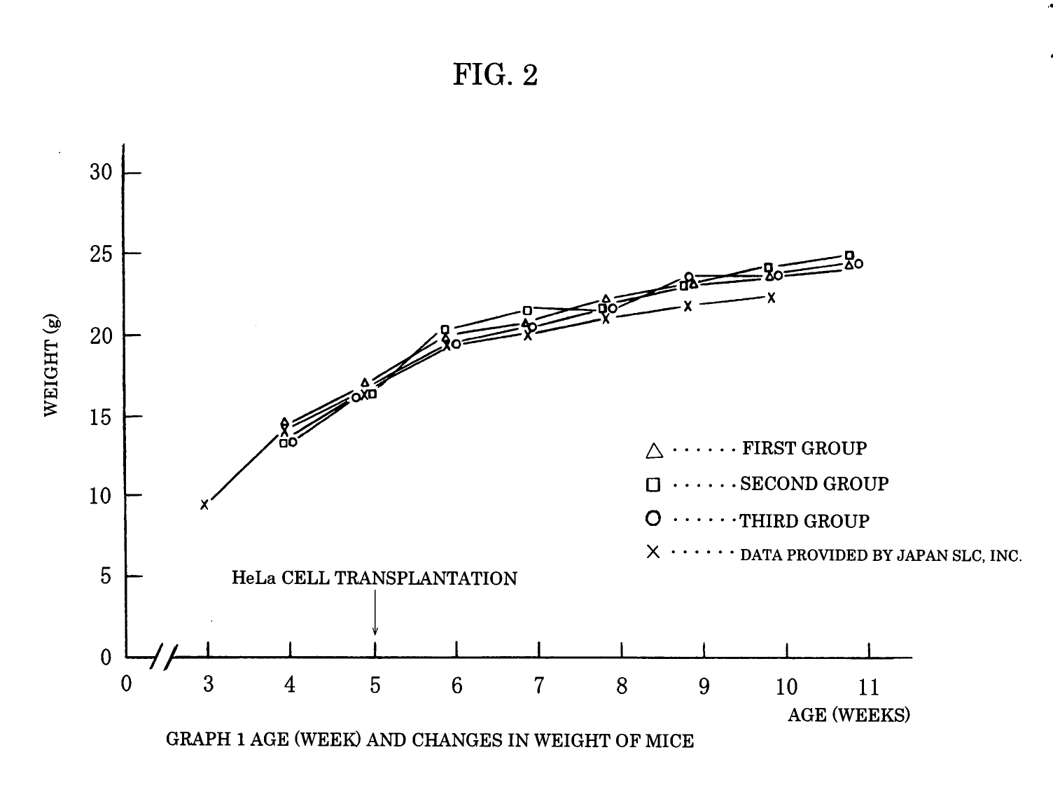

FIG. 2 (graph 1) shows changes in weight of the mice.

For comparison, data provided by Japan SLC, Inc. was plotted by “x”.

As a result, no significant difference was observed for the changes in weight of the mice.

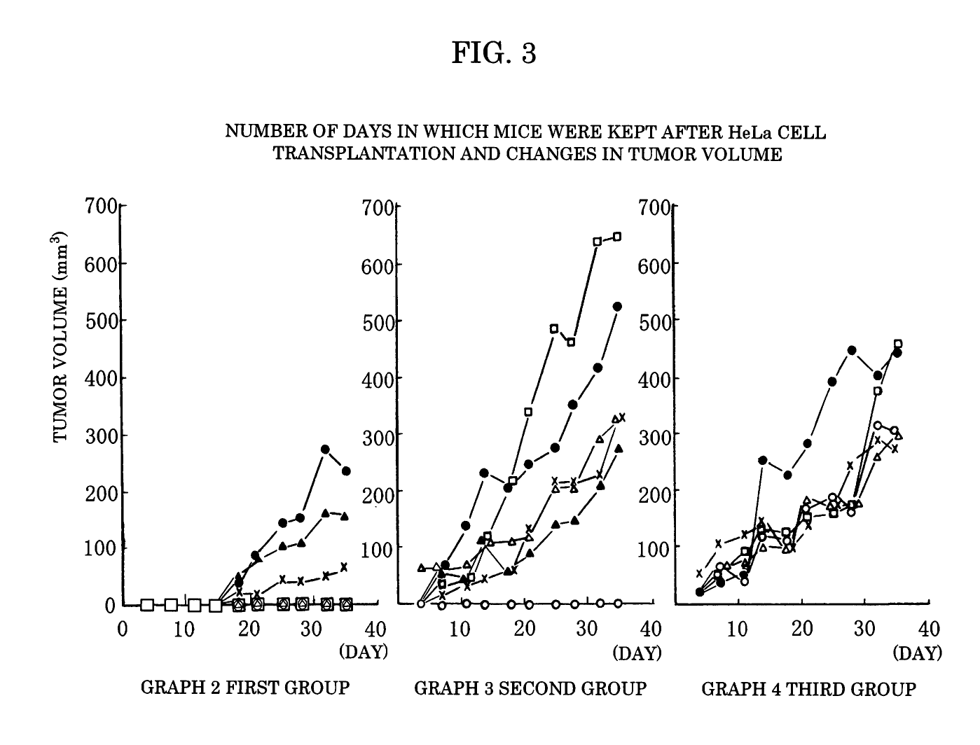

FIG. 3 (graphs 2, 3, and 4) show measurement results of the tumor size of each nude mouse in each group.

The major diameter “a” and the minor diameter “b” of the tumor were measured using calipers, and the tumor volume was calculated as indicated by “V=4/3ab2π”.

The first group and the second group respectively consisted of six mice, and the third group consisted of five mice. A tumor was not recognized in three mice in the first group (anticancer drug test solution), and a tumor occurred in the remaining three mice about 12 days after the HeLa cell transplantation. The first to third data (first to twelfth day) from the left in the graph 2 indicates that the data of the six mice overlaps at “0”, a tumor occurred in three mice at the fourth data (fifteenth day), and the data of the remaining three mice overlaps at “0” after the fifteenth day.

On the other hand, the tumor size increased in the five mice in the third group which were administered the normal water.

In the second group in which the mice were administered the test solution sterilized by filtration, while a tumor was not recognized in one mouse, the remaining five mice showed an increase in the tumor size. This suggests that the effects of the test solution sterilized by filtration do not significantly differ from those of the normal water (third group).

Specifically, significant anticancer effects were recognized for the first group in comparison with the second group (anticancer drug test solution sterilized by filtration) and the third group (normal water).

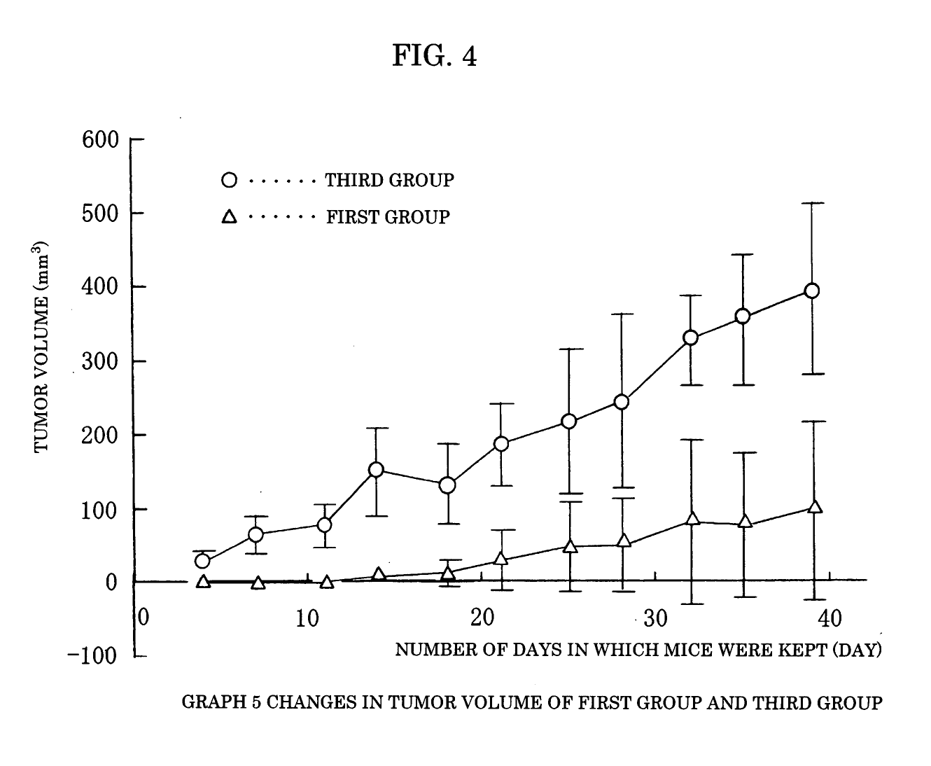

FIG. 4 (graph 5) shows changes in the average value (n=5 to 6) and the standard deviation of the tumor volume of the first group and the third group.

This graph clearly indicates that the deep sea water exhibits anticancer effects.

The reasons why the deep sea water sterilized by filtration does not exhibit the anticancer effects must be investigated in the future.

According to the invention, it was found that the deep sea water exhibits anticancer effects. Therefore, it is expected that the deep sea water will be applied to an anticancer drug.

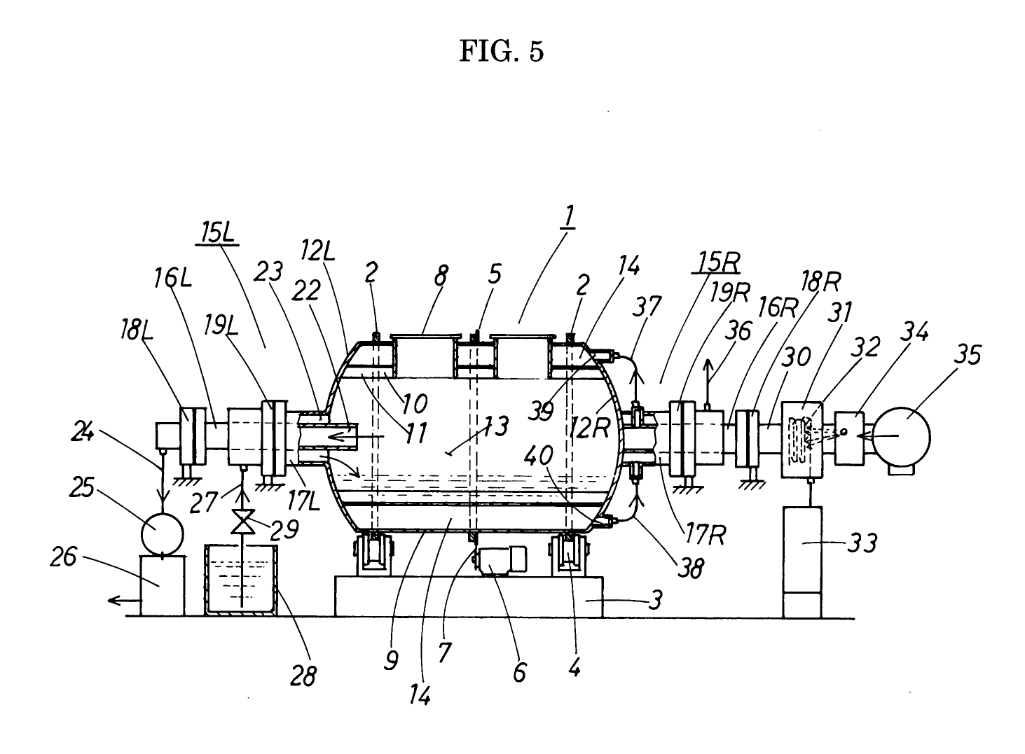

The method and apparatus for concentrating the deep sea water for obtaining the anticancer drug according to the invention are described below with reference to FIGS. 5 to 7.

FIG. 5 is a schematic view showing an embodiment of an apparatus for concentrating deep sea water. A rotary drum 1 constituting a processing vessel is formed in a cylindrical shape and provided with supporting flanges 2 at outer peripheral portions on both sides thereof and is rotatably supported by a base 3 freely around a horizontal axis by supporting two portions in the circumference of each of the supporting flanges 2 by a supporting roller 4 above the base 3. A ring-like sprocket 5 is fixed to a central portion of an outer periphery of the rotary drum 1 and the sprocket 5 and a sprocket (notation is not attached) attached to an output shaft of a motor 6 are connected by a chain 7 for driving the rotary drum 1.

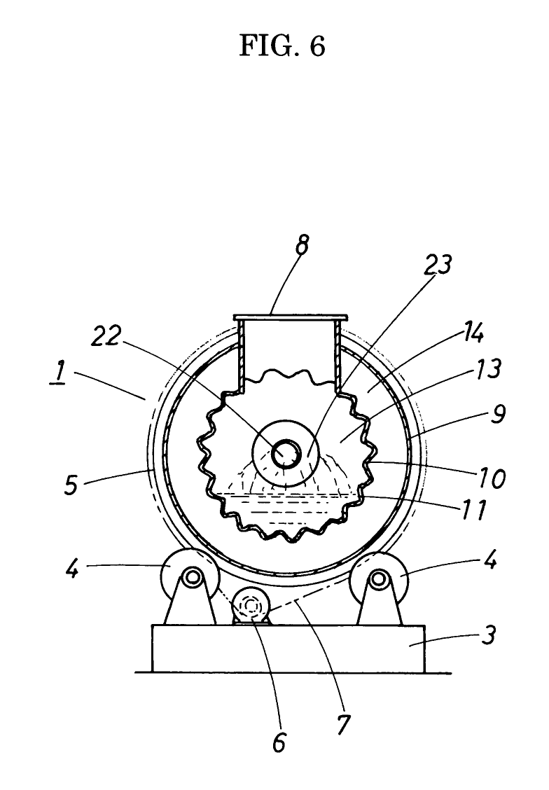

The rotary drum 1 is provided with opening and closing doors 8 fitted with transparent glass at several portions of the outer periphery. On an inner side of an outer peripheral wall 9 there is provided a heat conducting wall 1 formed with a number of folds 10 in an axial direction by being bent into a deep waveform as shown by FIG. 6. As shown by FIG. 5, the heat conducting wall 11 is arranged by fixing both ends thereof to panel boards 12L and 12R of the rotary drum 1 in airtight. As a result, an inner portion of the rotary drum 1 is partitioned into a decompression chamber 13 on an inner side of the heat conducting wall 11 and a steam chamber 14 on an outer side thereof.

Centers of the panel boards 12L and 12R on the both sides of the rotary drum 1 are respectively fixed with double tubes 15L and 15R and end portions of inner tubes 16L and 16R and outer tubes 17L and 17R of the respective double tubes 15L and 15R, are respectively connected with rotary couplings 18L and 19L, 18R and 19R. A base end of the inner tube 16L of the double tube on the left side of FIG. 5, constitutes a vacuum-suction port 22 projected to open to inside of the decompression chamber 13 and a base end of the outer tube 17L constitutes a sea water injecting port 23 opened in a ring-like shape at an inner face of the panel board 12L. The vacuum-suction port 22 is connected to a vacuum pump 25 via the rotary coupling 18L and a vacuum pipe 24. A condenser 26 is connected to a delivery side of the vacuum pump 25. The sea water injecting port 23 is connected to a sea water tank 28 via the rotary coupling 19L and a sea water pipe 27. The sea water pipe 27 is provided with an opening and closing valve 29.

The rotary coupling 18R is connected to a steam supply pipe 30 made of a nonconductive material of, for example, ceramic. The steam supply pipe 30 is provided with a high-frequency heater 31 and a high-frequency coil 32 wound around the steel supply pipe 30 is connected to a high-frequency power source 33. A water spray apparatus 34 for spraying water into the steam supply pipe 30, is provided in the steam supply pipe 30 on the front side (upstream side of steam flow) of the high-frequency heater 31 and a pushing fan 35 is provided on a further upstream side thereof. A steam discharging pipe 36 is connected to the rotor coupling 19R of the outer tube 17R of the double tube on the right side of FIG. 5. The inner tube 16R and the outer tube 17R are opened to an end portion of the steam chamber 14 via connection tubes 37 and 38 extended in the radius direction as a supply port 39 and a discharge port 40.

Next, an explanation will be given of a method of concentrating deep sea water which is carried out by using the above-described apparatus of the embodiment. Deep sea water sampled from deep sea is charged into the sea water tank 28. Inside of the decompression chamber 13 is vacuumed by operating the vacuum pump 25 and the condenser 26. Further, the pushing fan 35 is operated, high-frequency current is made to flow to the coil 32 of the high-frequency heater 31 and water is sprayed from the water spray apparatus 34. The sprayed water is heated by high frequency and is made to flow into the steam chamber 14 by the pushing fan 35 along with air.

By providing the high-frequency heater 31 having a sufficient capacity, the sprayed water is superheated to about 200° C. under ordinary pressure. When the opening and closing valve 29 is opened under the state, deep sea water in the sea water tank 28 is sucked into the decompression chamber 13. When a pertinent amount of deep sea water is sucked, the opening and closing valve 29 is closed and the rotary drum 1 is continuously rotated. Water in deep sea water in the decompression chamber 13, is evaporated by being heated by the heat conducting wall 11 heated by superheated steam of the steam chamber 14 and sucked by the vacuum pump 25 and condensed in the condenser 26. Deep sea water is supplied into the decompression chamber 13 by pertinently opening and closing the opening and closing valve 29 to replenish evaporated water. When the sea water tank 28 is emptied and deep sea water in the decompression chamber 13 is condensed, the apparatus is stopped.

Various kinds of compounds dissolving in deep sea water remain in the decompression chamber 13 in a condensed state and therefore, the compounds are vacuumed to recover by opening the opening and closing door 8. The recovered concentrated deep sea water is pertinently diluted and used as raw material of condiment, health food and addition solution of cosmetics. Further, the obtained concentrated deep sea water is put into a heating vessel and heated further by a gas stove or the like to thereby provide salt by evaporating the water. The obtained salt is used as raw material of condiment. Distilled water condensed in the condenser 26 is used as raw material of soft drink water or cosmetic water as the desalinated deep sea water.

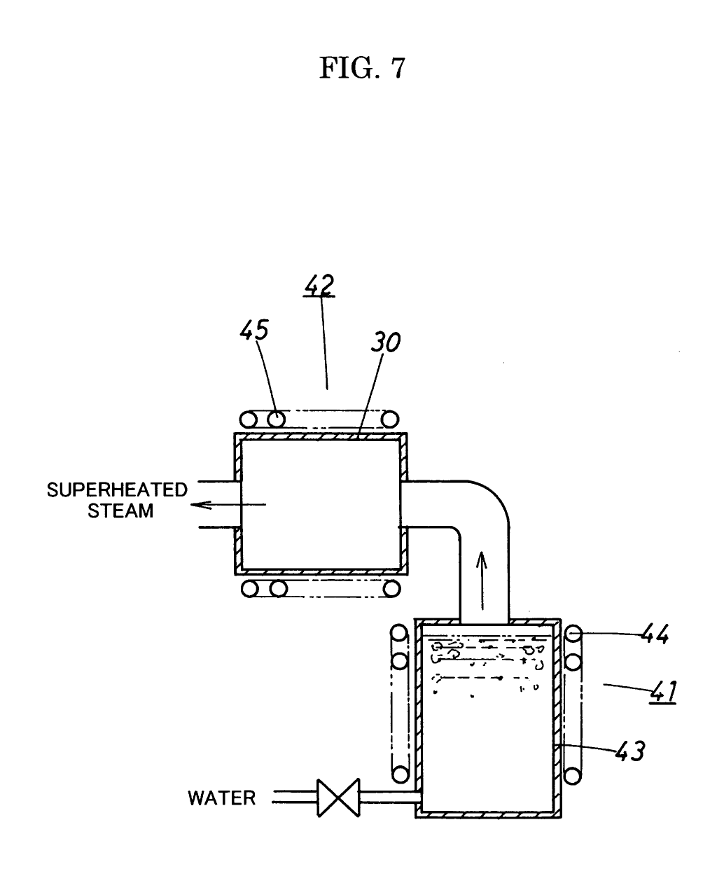

FIG. 7 is a view showing other apparatus for providing superheated steam used in the method of the invention. The apparatus of FIG. 7 is provided with a boiler 41 for obtaining saturated steam and a superheating apparatus 42 of the obtained saturated steam. The boiler 41 is constituted by winding a high-frequency coil 44 at a surrounding of a drum 43 made of a metal at an interval therebetween. By putting water to the drum 43 and applying high-frequency current to the coil 44, water is boiled by transmitting heat of an inductively heated drum wall to water in the drum to thereby generate saturated steam. The superheating apparatus 42 is constituted by winding a high-frequency coil 45 at a surrounding of a steam supply tube 30 made of ceramic for passing saturated steam. The saturated steam generated by the boiler 41 is inductively heated by the high-frequency coil 45 during a time period of passing the vapor supply tube 30 to constitute superheated steam and supplied to the steam chamber.

According to the embodiment explained above, solute contents in fresh water obtained by evaporating and condensing deep sea water and remaining concentrated sea water can be adjusted, and desalinated deep sea water and concentrated deep sea water with a rate of a mineral content changed in accordance with use can easily be obtained. Further, by using superheated steam heated by high frequency as heating means for distilling or condensing deep sea water, there is achieved an effect capable of carrying out swift processing by promoting a heat conduction efficiency, capable of providing the compact processing apparatus inexpensively and capable of carrying out efficient desalinating or condensing processing at low cost.

The invention is not limited to the above-described embodiment, and various modifications can be made within the scope of the invention. For example, the inner tube 16L of the double tube 15L shown in FIG. 5 is used for steam suction and the outer tube 17L of the double tube 15L is used for deep sea water supply in this embodiment, but the inner tube 16L may be used for deep sea water supply and the outer tube 17L may be used for steam suction. It is preferable to use a tube having less cross-sectional area for deep sea water supply to a decompression chamber, and a tube having larger cross-sectional area for steam suction.

What is claimed is:

1. An anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %.

2. An anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %,
wherein the concentrated sea water is obtained by a method of concentrating the deep sea water comprising steps of:
injecting the deep sea water into a decompression chamber;
evaporating water in the deep sea water by heating the deep sea water in the decompression chamber by supplying steam at an atmospheric pressure superheated to a temperature equal to or higher than a boiling temperature by high-frequency heating to a steam chamber formed around the decompression chamber with a heat conducting wall interposed therebetween; and
obtaining the concentrated sea water remaining in the decompression chamber.

3. An anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %,
wherein the concentrated sea water is obtained by a method of concentrating the deep sea water comprising steps of:
injecting the deep sea water into a decompression chamber;
obtaining steam by spraying air, which passes through a high-frequency coil, with water.
evaporating water in the deep sea water by heating the deep sea water in the decompression chamber by supplying the steam at an atmospheric pressure superheated to a temperature equal to or higher than a boiling temperature to a steam chamber formed around the decompression chamber with a heat conducting wall interposed therebetween; and
obtaining the concentrated sea water remaining in the decompression chamber.

4. An anticancer drug comprising, as a raw material, concentrated sea water obtained by concentrating deep sea water collected from a depth of 200 meters or more in the Sea of Japan under reduced pressure so that the deep sea water has a sodium chloride content of 15 to 18 mass %,
wherein the concentrated sea water is obtained by a method of concentrating the deep sea water comprising steps of:
injecting the deep sea water into a decompression chamber;
obtaining steam by passing saturated steam, which is produced by a boiler, through a steam pipe wound with a high-frequency coil.
evaporating water in the deep sea water by heating the deep sea water in the decompression chamber by supplying the steam at an atmospheric pressure superheated to a temperature equal to or higher than a boiling temperature to a steam chamber formed around the decompression chamber with a heat conducting wall interposed therebetween; and
obtaining the concentrated sea water remaining in the decompression chamber.
